(19) United States
(12) Patent Application Publication
(10) Pub. No.: US 2009/0298817 A1
(43) Pub. Date: Dec. 3, 2009

(54) ANTI-AMNESIC COMPOUNDS AND PHARMACEUTICAL COMPOSITIONS COMPRISING THEM

(76) Inventors: Marc Verleye, Remy (FR); Marie-Emmanuelle Le Guern, Compiegne (FR); Jean-Marie Gillardin, Jonquieres (FR); Bernard Hublot, Compiegne (FR)

Correspondence Address: STITES & HARBISON PLLC, 1199 NORTH FAIRFAX STREET, SUITE 900, ALEXANDRIA, VA, 22314, US

(21) Appl. No.: 12/436,257

(22) Filed: May 6, 2009

(30) Foreign Application Priority Data
May 6, 2008 (FR) 08 53003

(51) Int. Cl. A61K 31/536; A61P 25/28

(52) U.S. Cl. 514/230.5

(57) ABSTRACT

The present invention relates to the use of at least one compound of formula (I) as follows:

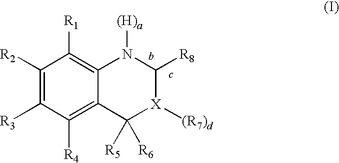

or of at least one pharmaceutically acceptable salt thereof
for the preparation of a drug for the prevention or treatment of memory disorders.

### FIELD OF THE INVENTION

The present invention relates to the use of novel anti-amnesic compounds in connection with the prevention or treatment of memory disorders.

### BACKGROUND OF THE INVENTION

The etiology of memory disorders is particularly varied.

Thus, the most common pathologies which involve a memory disorder include dementias, such as Alzheimer's disease, cerebral traumas, cerebral infections, such as encephalitis or meningitis, as well as cerebrovascular, ischemic or hemorrhagic strokes. The memory disorder may be linked to a biochemical deficiency, as in the case of Wernicke-Korsakov syndrome which is due to a serious vitamin B deficiency caused by chronic alcoholism. The memory disorder may also be of iatrogenic origin, and may in particular be induced by particular narcotic and/or pharmacologically active substances, such as sedatives, benzodiazepines, and related substances used as soporifics, antinauseants and anti-vertigo agents, because of the neuroleptic action thereof, anticholinergic antidepressants, centrally acting antihypertensives, or beta-blockers which cross the hematoencephalic barrier.

Memory disorders encompass in particular the various types of amnesia.

The most common is anterograde amnesia, characterized by an inability to store, retain or recall new knowledge after the event triggering the amnesia. This type of amnesia is observed in particular in patients suffering from dementia or Alzheimer's disease.

Retrograde amnesia, on the other hand, involves the loss of memories acquired before the onset of the disorder. It is often observed after cerebral trauma.

Finally, transient global amnesia is a temporary total loss of memory, with an inability to form new memories accompanied by a moderate loss of past memories. This rare variant, which regresses spontaneously, is observed primarily in the elderly but may also be caused by migraines, vascular strokes to the temporal lobe, or transient ischemic attacks.

The possible treatments for memory disorders generally depend on the cause of the disorder.

So, in the case of Korsakov syndrome, high-dosage vitamin B1 injections make it possible to reduce the memory deficit which is characteristic of this syndrome.

In contrast, memory disorders caused by Alzheimer's disease may be partially reduced using donepezil and drugs which improve the brain's cholinergic function. Two international applications also mention the development of peptides or polypeptides which are effective in treating memory disorders linked to Alzheimer's disease, either by acting on the amnesic effects of the β-amyloid protein (WO 1995/008999) or by acting as agonists to G protein receptors (WO 1996/039439).

Various studies have also been carried out to determine methods of treating post-traumatic memory disorders. Thus, a combined treatment of physostigmine and lecithin may improve the storage and recovery capacities for words in verbal memory (Goldberg et al. (1982) J. Clin. Neuropsychol. 4:219-234). It was also found that donepezil improved the memory of two amnesic patients after cerebral trauma (Taverni et al., (1998) Brain Injury 12:77-80).

Finally, there have been a number of studies relating to iatrogenic memory disorders. These memory disorders were thus reversed by virtue of a combined treatment with tacrine and galantamine, and reduced by piracetam or dihydroergocristine (Chopin and Briley, (1992) Psychopharmacology 106:26-30). A positive effect is also observed with aceclidine (Mashkovskii et al., (1997) Rev. Bull. Exp. Biol. Med. 123:296-298). More generally, galantamine may be used for all memory disorders caused by psychotropic substances (EP 1383507).

However, these different agents are not effective in all these situations. Moreover, they are responsible for a number of undesirable effects, in particular digestive problems.

The present invention relates to the provision of pharmaceutical compositions which can be used for the prevention or treatment of the various types of memory disorder.

Etifoxine, or 6-chloro-2-ethylamino-4-methyl-4-phenyl-4H-[3,1]benzoxazine hydrochloride, belongs to the benzoxazine family. It promotes gabaergic transmission by bonding to the site of the chloride channel coupled to the GABA(A) receptor, and is currently used as an anxiolytic. There are few undesirable effects reported subsequent to the use thereof.

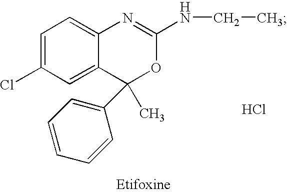

The synthesis of this compound is disclosed in particular in French patent No. 1 571 287. Moreover, a plurality of active metabolites of etifoxine were disclosed, such as desethyl etifoxine or 2-amino-6-chloro-4-methyl-4-phenyl-4H-[3,1]benzoxazine, 6-chloro-4-(4-hydroxyphenyl)-4-methyl-3,4-dihydro-1H-quinazolin-2-one or 6-chloro-3-ethyl-7-hydroxy-4-methyl-4-phenyl-3,4-dihydro-1H-quinazolin-2-one.

### SUMMARY OF THE INVENTION

The present invention is based on the finding, by the inventors, that etifoxine exhibits anti-amnesic action in animals in a model where memory disorder is induced by scopolamine.

The present invention thus relates to the use of at least one compound of formula (I) as follows:

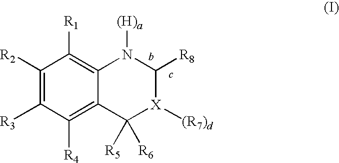

wherein:
a represents 0 or 1;
b represents a single bond or a double bond;
c represents a single bond or a double bond;
d represents 0 or 1;
X represents an oxygen or nitrogen atom, with the condition that if X represents an oxygen atom then d is equal to 0 and if X represents a nitrogen atom then d is equal to 1;
R₁, R₂, R₃, R₄ are the same or different and represent a hydrogen atom, a halogen atom, selected in particular from F, Cl, Br or I, a hydroxy group, or an alkoxy group with 1 or 2 carbon atoms;
R₅ and R₆ are the same or different and represent a hydrogen atom, an alkyl or cycloalkyl group with 1 to 6 carbon atoms, or an aryl group with 6 carbon atoms of which the aromatic ring is optionally substituted by one or more halogen atoms or one or more hydroxy groups, alkoxy groups with 1 or 2 carbon atoms, trifluoromethyl groups or nitro groups;
R₇ represents a hydrogen atom, a hydroxy group, or an alkyl or hydroxyalkyl group with 1 to 3 carbon atoms;
R₈ represents an oxygen atom or an —NR₉R₁₀ group, R₉ and R₁₀ being the same or different and representing a hydrogen atom, a hydroxy group, or an alkyl or hydroxyalkyl group with 1 to 3 carbon atoms, with the condition that if R₈ represents an oxygen atom then a is equal to 1, b represents a single bond and c represents a double bond and that if R₈ represents an —NR₉R₁₀ group then a is equal to 0, b represents a double bond and c represents a single bond;
or a pharmaceutically acceptable salt thereof,
for the preparation of a drug for the prevention or treatment of memory disorders.

The present invention also relates to a compound of formula (I) as defined above, or a pharmaceutically acceptable salt thereof, as a drug for the prevention or treatment of memory disorders.

The present invention also relates to a method for the prevention or treatment of memory disorder in an individual, comprising the administration to the individual of at least one compound of formula (I) as defined above, or a pharmaceutically acceptable salt thereof, in particular in a prophylactically or therapeutically effective amount.

In a particular embodiment of the invention, the compound of formula (I) is associated with at least one additional compound for the prevention or treatment of a pathology associated with or giving rise to a memory disorder.

The present invention also relates to a pharmaceutical composition comprising as an active substance:
at least one compound of formula (I) as defined above or a pharmaceutically acceptable salt thereof, and
at least one additional compound for the prevention or treatment of a pathology associated with or giving rise to a memory disorder,
in combination with a pharmaceutically acceptable excipient.

The present invention also relates to products containing:
at least one compound of formula (I) as defined above, or a pharmaceutically acceptable salt thereof, and
at least one additional compound for the prevention or treatment of a pathology associated with or giving rise to a memory disorder,
as a combined product for separate use, simultaneous use or use phased over time for the prevention or treatment of memory disorders.

### DETAILED DESCRIPTION OF THE INVENTION

### Compounds of Formula (I)

Compounds of formula (I) as defined above may be synthesized in a simple manner, based on the teaching of French patent No. 1 571 287.

The pharmaceutically acceptable salts according to the invention will then be obvious to the person skilled in the art, the hydrochloride salts of the compounds of formula (I) according to the invention being preferred in particular.

The invention also encompasses the optically active forms of the compound of formula (I) according to the invention, such as the following enantiomers (where R₅ and R₆ are different):

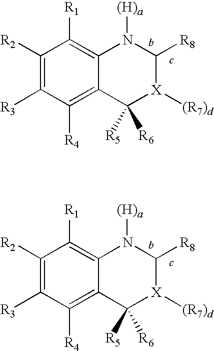

or mixtures thereof, in particular the racemic mixture thereof.

Preferably, the compound of formula (I) according to the invention is the compound of formula (VIII) as follows:

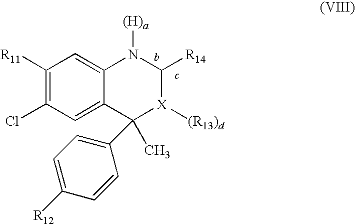

wherein:
a represents 0 or 1;
b represents a single bond or a double bond;
c represents a single bond or a double bond;
d represents 0 or 1;
X represents an oxygen or nitrogen atom, with the condition that if X represents an oxygen atom then d is equal to 0 and that if X represents a nitrogen atom then d is equal to 1;
R₁₁ and R₁₂ are the same or different and represent —H or —OH;
R₁₃ represents —H or a —CH₂—CH₃ group;
R₁₄ represents an oxygen atom or an —NH₂ or —NH—CH₂—CH₃ group, with the condition that if R₁₄ represents an oxygen atom then a is equal to 1, b represents a single bond and c represents a double bond and that if R₁₄ represents an —NH₂ or —NH—CH₂—CH₃ group then a is equal to 0, b represents a double bond and c represents a single bond.

The invention also encompasses the optically active forms of the compound of formula (VIII) according to the invention, such as the following enantiomers:

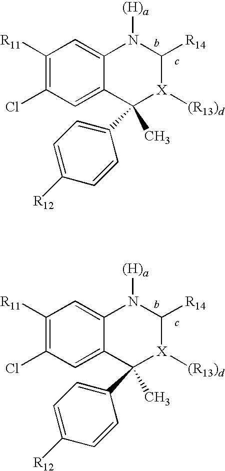

or mixtures thereof, in particular the racemic mixture thereof.

Preferably, the compound of formula (I) or (VIII) according to the invention is the compound of formula (II) as follows:

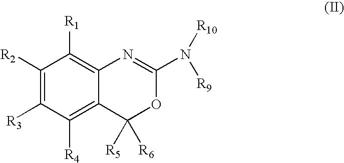

wherein R₁, R₂, R₃, R₄, R₅, R₆, R₉ and R₁₀ are as defined above.

The invention also encompasses the optically active forms of the compound of formula (II), such as the following enantiomers (where R₅ and R₆ are different):

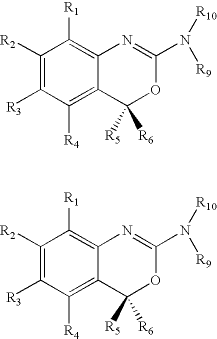

or mixtures thereof, in particular the racemic mixture thereof.

The compound of formula (I), (II) or (VIII), according to the invention, is particularly preferably represented by the compound of formula (III) or (IV) as follows:

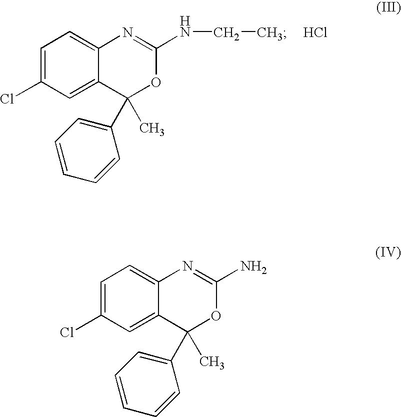

The compound of formula (III) is etifoxine, or 6-chloro-2-ethylamino-4-methyl-4-phenyl-4H-[3,1]benzoxazine hydrochloride.

The compound of formula (IV), desethyl etifoxine or 2-amino-6-chloro-4-methyl-4-phenyl-4H-[3,1]benzoxazine, is a metabolite of etifoxine.

The invention also encompasses the optically active forms of the compound of formula (III) according to the invention, such as the following enantiomers:

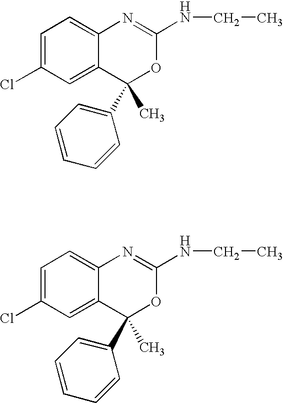

or mixtures thereof, in particular the racemic mixture thereof, in particular in the form of the hydrochloride, as well as the optically active forms of the compound of formula (IV) according to the invention, such as the following enantiomers:

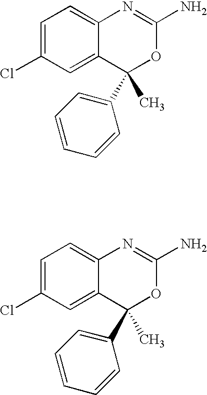

or mixtures thereof, in particular the racemic mixture thereof.

Preferably, the compound of formula (I) or (VIII), according to the invention, is the compound of formula (V) as follows:

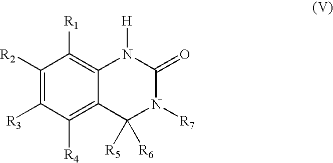

wherein R₁, R₂, R₃, R₄, R₅, R₆ and R₇ are as defined above.

The invention also encompasses the optically active forms of the compound of formula (V) according to the invention, such as the following enantiomers (where R₅ and R₆ are different):

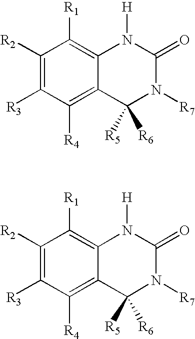

or mixtures thereof, in particular the racemic mixture thereof.

The compound of formula (I), (V) or (VIII), according to the invention, is particularly preferably represented by the compound of formula (VI) or (VII) as follows:

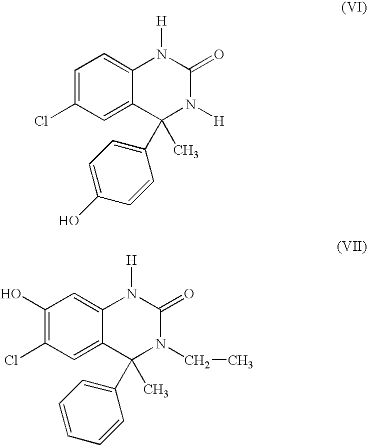

The compounds of formula (VI) (6-chloro-4-(4-hydroxy-phenyl)-4-methyl-3,4-dihydro-1H-quinazolin-2-one) and (VII) (6-chloro-3-ethyl-7-hydroxy-4-methyl-4-phenyl-3,4-dihydro-1H-quinazolin-2-one) are metabolites of etifoxine.

The invention also encompasses the optically active forms of the compound of formula (VI), such as the following enantiomers:

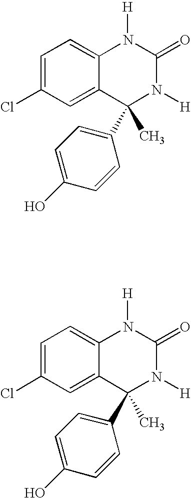

or mixtures thereof, in particular the racemic mixture thereof, as well as the optically active forms of the compound of formula (VII), such as the following enantiomers:

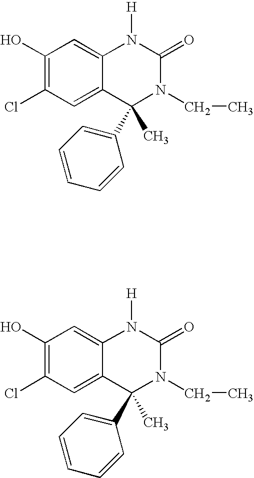

or mixtures thereof, in particular the racemic mixture thereof.

### Memory Disorders

An individual is said to be suffering from a “memory disorder” if he has a deficit in his ability to learn new information or an inability to remember information learnt or events which took place before the onset of the disorder.

Memory disorders are in particular defined on pages 123 to 163 of the reference manual “Diagnostic and Statistical Manual IV” (DSM IV) in relation to the pathologies of delirium, dementias and amnesic disorders.

Amnesic disorders, which are therefore a subset of memory disorders, are in particular characterized by a deterioration of the memory without any other significant cognitive deteriorations. The classification thereof is based on the suspected etiology, as these disorders may be caused by a general medical affliction, be induced by a substance, or be non-specific.

Thus, memory disorders are in the present case to be understood as comprising all forms of amnesia, in particular anterograde amnesia, retrograde amnesia, transient global amnesia, amnesic stroke or even pure amnesic syndrome.

The memory disorders according to the invention may in particular be due to the action of exogenous substances (i.e. the memory disorder is of iatrogenic origin) or be due to organic lesions. These lesions may in particular be of a degenerative, ischemic, hemorrhagic or traumatic type or be due to deficiencies, for example of vitamins. The non-demential etiology of memory disorders is summarized in particular by Michel & Sellal (2006) Le Concours Médical 128:487-491.

Thus, the memory disorders are preferentially linked to or caused by the pathologies selected from the list consisting of:
traumatic cerebral injury, such as a head trauma;
cerebral infection, such as meningitis or encephalitis, in particular limbic, herpetic or paraneoplastic;
neurodegenerative disease and/or dementia, such as Alzheimer's disease, in particular in its prodromal form or frontally expressed, Pick's disease, semantic dementia, frontotemporal dementia, corticobasal dementia, diffuse Lewy body dementia, Huntingdon's chorea, Creutzfeldt-Jakob disease, posterior cortical atrophy, or multiple system atrophy (MSA);
a demyelinising affliction, such as multiple sclerosis;
cerebrovascular, ischemic or hemorrhagic stroke, such as a hippocampal infarction, chronic subdural hematoma, cerebral anoxia,
an epileptic fit;
a cerebral tumor;
a state of deficiency, such as Gayet-Wernicke encephalopathy or Korsakov syndrome;
an iatrogenic memory disorder, in particular due to the administration of pharmacological compounds such as sedatives, benzodiazepines and related substances, antinauseants and anti-vertigo agents, anticholinergic antidepressants, centrally acting antihypertensives, corticosteroids or beta-blockers which cross the hematoencephalic barrier
intoxication, in particular with alcohol, carbon monoxide, mercury, bismuth, lithium or lead (saturnism);
organ failure, such as chronic liver failure or thyroid failure;
paralysis, such as general paralysis, supranuclear paralysis, primary progressive apraxia, frontal progressive anarthria, or progressive aphasia;
a behavioral disorder, such as an anxiety disorder, major depressive disorder, or stress;
hydroelectrolytic or metabolic disorder.

More preferentially, the memory disorders are iatrogenic memory disorders, in particular due to the administration of pharmacological compounds such as sedatives, benzodiazepines and related substances, antinauseants and anti-vertigo agents, anticholinergic antidepressants, centrally acting antihypertensives, corticosteroids or beta-blockers which cross the hematoencephalic barrier.

### Additional Compound

As will be clear to the person skilled in the art, the additional compound is not a compound of formula (I) according to the invention.

Preferably, the additional compound is for the treatment of a pathology linked to or caused by a memory disorder selected from the list consisting of:
traumatic cerebral injury, such as a head trauma;
cerebral infection, such as meningitis or encephalitis, in particular limbic, herpetic or paraneoplastic;
neurodegenerative disease and/or dementia, such as Alzheimer's disease, in particular in its prodromal form or frontally expressed, Pick's disease, semantic dementia, frontotemporal dementia, corticobasal dementia, diffuse Lewy body dementia, Huntingdon's chorea, Creutzfeldt-Jakob disease, posterior cortical atrophy, or multiple system atrophy (MSA);
a demyelinising affliction, such as multiple sclerosis;
cerebrovascular, ischemic or hemorrhagic stroke, such as a hippocampal infarction, chronic subdural hematoma, cerebral anoxia,
an epileptic fit;
a cerebral tumor;
a state of deficiency, such as Gayet-Wernicke encephalopathy or Korsakov syndrome;
an iatrogenic memory disorder, in particular due to the administration of pharmacological compounds such as sedatives, benzodiazepines and related substances, antinauseants and anti-vertigo agents, anticholinergic antidepressants, centrally acting antihypertensives, corticosteroids or beta-blockers which cross the hematoencephalic barrier
intoxication, in particular with alcohol, carbon monoxide, mercury, bismuth, lithium or lead (saturnism);
organ failure, such as chronic liver failure or thyroid failure;
paralysis, such as general paralysis, supranuclear paralysis, primary progressive apraxia, frontal progressive anarthria, or progressive aphasia;
a behavioral disorder, such as an anxiety disorder, major depressive disorder, or stress;
hydroelectrolytic or metabolic disorder.

Moreover, the additional compound defined above is preferably selected from the list comprising or consisting of:
a compound for the treatment of Alzheimer's disease, in particular:
   an anticholinesterase, such as:
      donepezil,
      galantamine,
      rivastigmine,
   an NMDA receptor antagonist, such as:
      memantine,
a compound for the treatment of ischemic cerebrovascular strokes (CVS) in acute phase (altephase) and the consequences thereof (dihydroergocristine, piracetam), in particular, according to the CVS type:
   heparin,
   aspirin,
   nicardipine,
a compound for the treatment of cerebral hemorrhages, in particular:
   nimopidine,
an antidepressant, in particular:
   an imipramine-type drug, such as:
      imipramine,
      clomipramine,
      amoxapine,
      amitriptyline,
   a selective serotonin reuptake inhibitor, such as:
      fluoxetine,
      paroxetine,
      citalopram,
      fluvoxamine,
   a selective serotonin and noradrenaline reuptake inhibitor, such as:
      venlafaxine,
      mirtazapine,
      tianeptine,
a compound for the treatment of cerebral tumors, in particular a chemotherapy agent,
a compound for the treatment of alcoholic polyneuropathy, in particular vitamin B1.

### Administration

Preferably, the drug, pharmaceutical composition or product according to the invention comprises a single dose of approximately 25 mg to approximately 2500 mg, in particular approximately 100 to 1000 mg of the compound of formula (I) as defined above, or the pharmaceutically acceptable salt thereof.

It is also preferable that the compound of formula (I) as defined above, or the pharmaceutically acceptable salt thereof, is administered in a dose of approximately 25 mg/day to approximately 2500 mg/day, in particular approximately 100 mg/day to approximately 1000 mg/day.

Preferably, the drug, pharmaceutical composition or product according to the invention is suitable for oral administration.

It is also preferable that the drug, pharmaceutical composition or product according to the invention is in the form of a powder, wafers, capsules or sachets.

### DESCRIPTION OF THE FIGURES

FIG. 1: Histograms representing the mean latency time to enter the dark compartment in the course of information acquisition (hollow bars) or in the course of information retention (hatched bars) as a function of the different treatments performed. Scopolamine (SCOP) or its excipient (dose 0) were administered 30 minutes before acquisition, at a dose of 1.4 mg/kg. Etifoxine (EFX-mg/kg) was administered 35 minutes before acquisition, at the doses indicated.

FIG. 2: Histograms representing the mean latency time to enter the dark compartment in the course of information acquisition (hollow bars) or in the course of information retention (hatched bars) as a function of the different treatments performed. Scopolamine (SCOP) or its excipient (dose 0) were administered 30 minutes before acquisition, at a dose of 1.4 mg/kg. Etifoxine (EFX-mg/kg) was administered immediately after acquisition, at the doses indicated.

### EXAMPLE

### Study of the Effects of Etifoxine in a Model Where Amnesia is Induced by Scopolamine in Rats

### Aim

The purpose of the study is to examine the effects of etifoxine on amnesia, induced by a substance which is an antagonist of the cholinergic receptors in rats, scopolamine (see in particular Rush (1988) Behav. Neural. Biol. 1988 50:255-274).

### Method

Memory capacities are evaluated over the course of the passive avoidance test in a box with two compartments, dark and light. In a first step, the acquisition step, entering the dark compartment is associated with an unpleasant electric shock. In a second step, the retention step, the latency to enter the dark compartment is measured. The lower this latency, the more strongly the animal is suffering from amnesia.

Amnesia is induced by administering scopolamine 30 minutes before the information acquisition step.

Two series of tests were performed:
1) Etifoxine was administered intraperitoneally 35 minutes before acquisition of the event.
7 groups of 10 to 20 rats were formed, each group receiving the following respective treatments:
Group 1 (reference): scopolamine 0 mg/kg, etifoxine 0 mg/kg
Group 2 (control): scopolamine 0 mg/kg, etifoxine 50 mg/kg
Group 3: scopolamine 1.4 mg/kg, etifoxine 0 mg/kg
Group 4: scopolamine 1.4 mg/kg, etifoxine 6.2 mg/kg
Group 5: scopolamine 1.4 mg/kg, etifoxine 12.5 mg/kg
Group 6: scopolamine 1.4 mg/kg, etifoxine 25 mg/kg
Group 7: scopolamine 1.4 mg/kg, etifoxine 50 mg/kg
2) Etifoxine was administered intraperitoneally immediately after acquisition of the event.
7 groups of 10 rats were formed, each group receiving the following respective treatments:
Group 1 (reference): scopolamine 0 mg/kg, etifoxine 0 mg/kg
Group 2 (control): scopolamine 0 mg/kg, etifoxine 50 mg/kg
Group 3: scopolamine 1.4 mg/kg, etifoxine 0 mg/kg
Group 4: scopolamine 1.4 mg/kg, etifoxine 6.2 mg/kg
Group 5: scopolamine 1.4 mg/kg, etifoxine 12.5 mg/kg
Group 6: scopolamine 1.4 mg/kg, etifoxine 25 mg/kg
Group 7: scopolamine 1.4 mg/kg, etifoxine 50 mg/kg

For each test series and for each group the latency to enter the dark compartment was measured in the case of acquisition and in the case of retention.

For the first series of tests, a one-way ANOVA analysis was performed followed by the Student-Newman-Keuls test in order to find the differences between the groups. The difference is considered significant if the critical value P obtained was less than 0.05.

For the second series of tests, the non-parametric Kruskal-Wallis test was performed followed by the Dunn test in order to find the differences between the groups. The difference is considered significant if the critical value P obtained was less than 0.05.

### Results

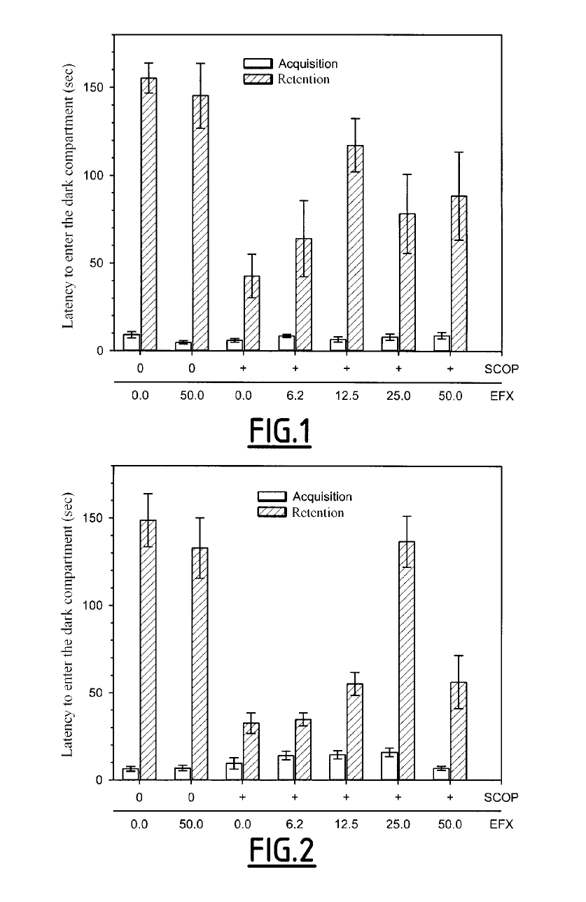

Scopolamine administered 30 minutes before acquisition triggers amnesia in rats, as manifested by the short time period to enter the dark compartment, which is significantly shorter than the time period observed in the reference groups (FIGS. 1, 2; (SCOP+, EFX 0) vs. (SCOP 0, EFX 0)).

In the two administration schemes, it is observed that etifoxine counteracts the amnesic effects of scopolamine according to an inverted U-shaped curve (FIG. 1, FIG. 2).

1. A method for the prevention or treatment of memory disorders in an individual, comprising administering the individual a prophylactically or therapeutically effective amount of at least one compound of the following formula (I): 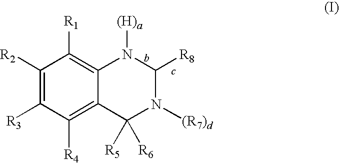
wherein:
a represents 0 or 1;
b represents a single bond or a double bond;
c represents a single bond or a double bond;
d represents 0 or 1;
X represents an oxygen or nitrogen atom, with the condition that if X represents an oxygen atom then d is equal to 0 and if X represents a nitrogen atom then d is equal to 1;
R₁, R₂, R₃, and R₄ are the same or different and represent a hydrogen atom, a halogen atom, selected in particular from F, Cl, Br or I, a hydroxy group, or an alkoxy group with 1 or 2 carbon atoms;
R₅ and R₆ are the same or different and represent a hydrogen atom, an alkyl or cycloalkyl group with 1 to 6 carbon atoms, or an aryl group with 6 carbon atoms of which the aromatic ring is optionally substituted by one or more halogen atoms or one or more hydroxy groups, alkoxy groups with 1 or 2 carbon atoms, trifluoromethyl groups or nitro groups;
R₇ represents a hydrogen atom, a hydroxy group, or an alkyl or hydroxyalkyl group with 1 to 3 carbon atoms;
R₈ represents an oxygen atom or an —NR₉R₁₀ group, R₉ and R₁₀ being the same or different and representing a hydrogen atom, a hydroxy group, or an alkyl or hydroxyalkyl group with 1 to 3 carbon atoms, with the condition that if R₈ represents an oxygen atom then a is equal to 1, b represents a single bond and c represents a double bond and that if R₈ represents an
N₉R₁₀ group then a is equal to 0, b represents a double bond and c represents a single bond;
or of at least one pharmaceutically acceptable salt thereof.

2. The method according to claim 1, wherein the at least one compound is of the following formula (II): 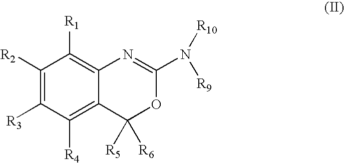
wherein R₁, R₂, R₃, R₄, R₅, R₆, R₉ and R₁₀ are as defined above.

3. The method according to claim 1, wherein the at least one compound is of the following formula (III) or (IV):

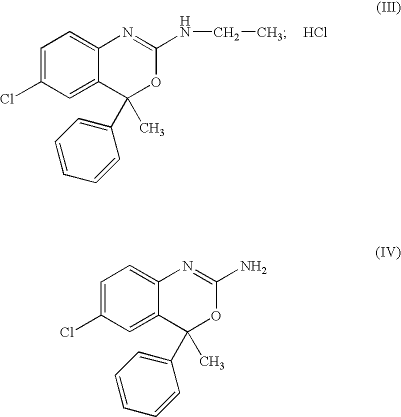

4. The method according to claim 1, wherein the at least one compound is of the following formula (V): 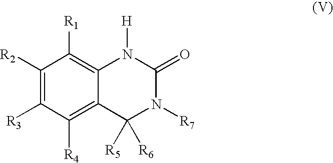
wherein R₁, R₂, R₃, R₄, R₅, R₆ and R₇ are as defined in claim 1.

5. The method according to claim 1, wherein the at least one compound is of the following formula (VI) or (VII):

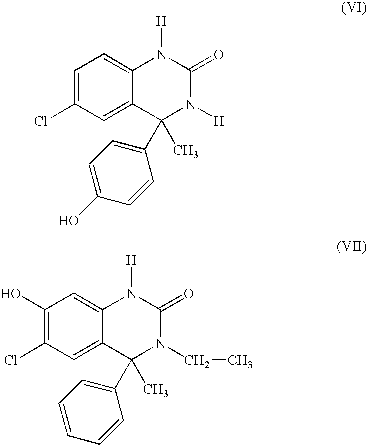

6. The method according to claim 1, wherein the at least one compound is administered in a single dose of approximately 25 mg to approximately 2500 mg, in particular of approximately 100 to 1000 mg.

7. The method according to claim 1, wherein the at least one compound is administered orally.

8. The method according to claim 1, wherein the at least one compound is in the form of a powder, wafers, capsules or sachets.

9. The method according to claim 1, wherein the at least one compound is associated with at least one additional compound for the prevention or treatment of a pathology associated with memory disorder.

10. A pharmaceutical composition comprising as an active substance:
at least one compound of formula (I) as defined in claim 1, or a pharmaceutically acceptable salt thereof, and
at least one additional compound for the prevention or treatment of a pathology associated with a memory disorder,
in combination with a pharmaceutically acceptable excipient.
